(19) United States
(12) Patent Application Publication
(10) Pub. No.: US 2014/0178939 A1
(43) Pub. Date: Jun. 26, 2014

(54) METHOD FOR DNA AMPLIFICATION BASED ON THE ORIGINS OF REPLICATION OF THE BACTERIOPHAGE P29 AND ASSOCIATED NUCLEOTIDE SEQUENCES

(71) Applicants: Margarita Salas Falgueras, Madrid (ES); Mario Mencia Caballerro, Madrid (ES); Miguel De Vega Jose, Madrid (ES); Jose M. Lazaro Bolos, Madrid (ES); Pablo Gella Montero, Madrid (ES)

(72) Inventors: Margarita Salas Falgueras, Madrid (ES); Mario Mencia Caballerro, Madrid (ES); Miguel De Vega Jose, Madrid (ES); Jose M. Lazaro Bolos, Madrid (ES); Pablo Gella Montero, Madrid (ES)

(73) Assignee: CONSEJO SUPERIOR DE ENVESTIGACIONES CIENTIFICAS (CSIC), Madrid (ES)

(21) Appl. No.: 14/002,644

(22) PCT Filed: Feb. 28, 2012
(86) PCT No.: PCT/ES2012/070121

§ 371 Date: Jan. 27, 2014

(30) Foreign Application Priority Data
Mar. 3, 2011 (ES) P201130288

(51) Int. Cl. C12P 19/34

(52) U.S. Cl. CPC C12P 19/34
USPC 435/91.2; 435/320.1; 536/24.1; 435/194

(57) ABSTRACT

The present invention refers to a DNA amplification method based on the origins of replication of bacteriophage φ29, and to the genic constructs, vectors and oligonucleotides that can be used in the method for amplifying an exogenous sequence of interest.

The present invention corresponds to the field of molecular biology and refers to a DNA amplification method based on the replication origins of bacteriophage φ29, and to the genic constructs, vectors and oligonucleotides that can be used in the method for amplifying an exogenous sequence of interest.

### STATE OF THE PREVIOUS TECHNIQUE

Deoxyribonucleic acid amplification methods are of great importance in molecular biology. The most widely used techniques are polymerase chain reaction (PCR) and multiple displacement amplification (MDA). Although PCR has the advantage of being very effective and generates defined DNA segments, it is not able to amplify sequences of more than 20 kilobases (kb) in length.

While the most common methods for starting DNA replication use a DNA or RNA molecule as primer, DNA synthesis can also be started using a protein as primer. As receptor of the first phosphodiester bond, the systems of this latter kind start DNA synthesis using the OH group of a specific serine, threonine or tyrosine residue present in a given protein, instead of the 3′ OH group of a ribose. This protein is generally referred to as the terminal protein (TP), since it is covalently bound to the 5′-extremity of the DNA molecule. Studies have been made of a broad series of DNA replication systems involving initiation with TP, such as certain bacteriophages (φ29, Nf, GA-1, PRD1 or Cp-1), linear plasmids from bacteria (pSCL and pSLA2), mitochondrial DNA, DNA from yeasts and plants, bacterial chromosomes (Streptomyces spp.), and DNA from mammalian viruses (adenovirus) (Salas, M. 1991. Annu. Rev. Biochem. 60, 39-71).

There have also been descriptions of in vivo methods for generating linear DNA chains with TP bound to the 5′ extremities for plasmids of Streptomyces (Shiffman, D. and Cohen, S. N. 1992. Proc Natl Acad Sci USA 89, 6129-33) and for adenovirus (Crouzet, J. et al., 1997 Proc Natl Acad Sci USA 94, 1414-9). These methods are based on the observation that following transformation and selection in the appropriate host, DNA containing the adequate sequences but lacking bound TP can acquire the TP needed for replication and stable maintenance within the cell. The obtainment of TP-DNA implies execution of the adequate cloning steps for the plasmid to generate the signals needed for replication based on the TP. DNA construction then must undergo a cloning and selection process in an adequate host, and finally the DNA must be extracted from the host, followed by purification for the intended application purposes. All these steps can limit the size and restrict the type of sequences that can be cloned, maintained and obtained in a stable manner. To date, none of these systems have been employed to produce substantial amounts of TP-DNA for different uses in molecular biology.

Regarding the in vitro approach, the most effective system is based on DNA replication of bacteriophage φ29. Gutierrez et al. (Nucleic Acids Research 1988. 16 (13); 5895-5914) described the analysis of the minimal origins of in vitro initiation and replication in the left and right extremities of the DNA of bacteriophage φ29, and found these minimal origins to comprise the 12 terminal nucleotides. Within these 12 nucleotides, the three terminal nucleotides cannot be mutated without reducing the efficiency of initiation and replication, while the sequence in less terminal positions does not have to be so exact. Descriptions have also been made of the in vitro initiation and replication of DNA based on the replication origins of φ29 in the presence of the DNA polymerase of bacteriophage φ29 and the TP of φ29, though not referred to the amplification of DNA.

WO9010064 describes a method for amplifying a DNA sequence of up to several hundred kb flanked by the replication origins of φ29—specifically, the 12 nucleotides of the left extremity of the DNA of φ29 on one side and the 12 nucleotides of the right extremity of the DNA of φ29 on the other, in the presence of the DNA polymerase of bacteriophage φ29 and the TP of φ29. WO9010064 describes oligonucleotides comprising the 12 nucleotides of the left extremity of the DNA of φ29. However, the method described in WO9010064 is based on the results of Gutiérrez et al. (1988), which are not referred to DNA amplification but to DNA replication. The results presented by Blanco et al. (Proc. Natl. Acad. Sci. USA. 1994. Vol. 91; 12198-12202) show that the DNA amplification described in WO9010064 is not possible; consequently, the description in this patent regarding DNA amplification is wrong, since DNA amplification cannot be carried out with only the 12 nucleotides of the extremity of the DNA of φ29.

Blanco et al. (1994) reported that efficient in vitro amplification of the DNA of φ29 requires the presence not only of the DNA polymerase of bacteriophage φ29 and the TP of φ29, but also of large amounts of proteins p5 and p6 of φ29.

The fact that TP is covalently bound to the genome of φ29 destined for amplification means that the amplification reaction will be much more efficient. It is not yet clear whether covalent bonding of TP to the extremities of the DNA of φ29 is essential for amplification of this DNA, or whether the same system could be used to amplify heterologous DNA lacking TP bound to its extremities.

### DESCRIPTION OF THE INVENTION

The start of replication, based on templates with extremities of the DNA of φ29 lacking TP, is much less effective than the start of replication based on the φ29 genome as template, since the genome, along with the adequate extremities, contains TP covalently bound to the 5′ phosphate of the extremities of both DNA strands.

The present invention describes something that has not been previously done and experimentally demonstrates in vitro DNA amplification, comparing that of the φ29 genome with TP covalently bound to its extremities (TP-DNA) versus other templates with terminal regions of the DNA of φ29, but without TP, using the minimum φ29 amplification machinery.

The authors of the present invention have found that in order for linear DNA to be amplified with the φ29 system, it must possess totally functional extremities of the DNA of φ29, distributed with the correct orientation and joined in a single DNA fragment.

The minimal origins of φ29 DNA replication described in this study do not require TP covalently bound to the 5′ extremities in order to be functional in amplification, and can bind to the DNA destined for amplification using different common methods in molecular biology, such as ligation, recombination or cloning.

The in vitro DNA replication and amplification process has been studied for decades, and it has been necessary to experimentally perform each reaction to firmly establish the minimum requirements in order to ensure that such replication and amplification is functional.

The present study describes a method for the isothermal and defined amplification of large DNA fragments based on their insertion between two DNA sequences containing the minimum replication origins of the DNA of bacteriophage φ29. This method generates products possessing TP covalently bound to the 5′ extremities of the amplified DNA, since TP is used as a “universal initiator” for all the amplifications made with this system.

The extremely high intrinsic processivity and capacity to couple polymerization to the band displacement of the DNA polymerase of φ29 make it possible to perform DNA amplifications of great length.

The resulting TP-DNA replicons can be used directly in applications in which the presence of TP is either of benefit or necessary, such as for example cell transformation, DNA transference or gene therapy in vivo, the in vitro evolution of proteins by DNA-display, bacterial transformation, sequencing, genotyping, etc. Alternatively, TP can be eliminated using different reactions known to experts in the field. Regarding paternal TP, it has been shown not to be absolutely essential for amplification. Paternal TP is the protein bound to the extremity of the parental DNA destined for replication or amplification. In order for replication or amplification to start, a new TP called the initiator TP must be incorporated (FIG. 1).

Therefore, a first aspect of the present invention refers to a genic construct adapted for the introduction of an exogenous nucleotide sequence, and which consists of at least a nucleotide sequence with the following three elements in the stated order:
a) at least a nucleotide sequence comprising a replication origin of the DNA of bacteriophage φ29, with a terminal extremity followed by:
b) at least one cleavage site of the nucleotide sequence,
c) at least a nucleotide sequence comprising a replication origin of the DNA of bacteriophage φ29 with an orientation opposite that of the nucleotide sequence in (a).

The term “genic construct”, as used in this study, refers to a DNA molecule that has been artificially assembled. The genic construct is adapted for the introduction of an exogenous nucleotide sequence, which may be any nucleotide sequence of interest.

The DNA of bacteriophage φ29 is linear and double stranded, and a replication origin is found at each of its extremities, referred to as left and right. The DNA replication origin of bacteriophage φ29 is the nucleotide sequence of one of the extremities of the genome of bacteriophage φ29, where replication of the mentioned genome starts. The terminal extremity of a replication origin is that where the TP binds.

A cleavage site is a region in the nucleotide sequence which as a result of its sequence is amenable to cleaving.

In a preferred approach to the first aspect of the invention, the nucleotide sequences of the three elements are juxtaposed or overlapped. It is more preferable for the nucleotide sequences of the three elements to be juxtaposed.

In a preferred approach to the first aspect of the invention, (b) is at least a nucleotide sequence comprising the cleavage target sequence of a restriction enzyme. Preferably, the restriction enzyme gives rise to blunt extremities or to protruding 3′ extremities. More preferably, it gives rise to blunt extremities.

A cleavage site of a nucleotide sequence can have different presentations. In addition to cleavage with restriction enzymes, a nucleotide sequence can be cleaved with repair enzymes, or by generating an abasic site followed by treatment of the nucleotide sequence with an apurinic/apyrimidinic (AP) endonucleases. The nucleotide sequence can also be cleaved by introducing a ribonucleotide and then eliminating it with alkaline treatment. Another cleaving or cutting option is to introduce two cleavage sites in the nucleotide sequence, one in each strand, for action upon of enzymes that produce single-strand incisions.

The φ29 amplification system based on the vector pETORPHIBae (SEQ ID NO: 3), presented in the examples of the present report can effectively generate amplification products from templates with simple band extensions of 5 nucleotides at the 3′ extremity.

In a preferred approach to the first aspect of the invention, the genic construct is a circular vector. The size of the genic construct is preferably between 4800 and 10,000 base pairs (bp).

A vector is a nucleic acid molecule used to transfer genetic material of interest to a cell. Apart from such genetic material, a vector can also contain different functional elements, including transcription control elements such as promoters or operators, transcription factor binding enhancers or regions, and control elements for starting and ending translation. Vectors include, but are not limited to the following: plasmids, cosmids, viruses, phages, recombinant expression cassettes and transposons. Some vectors are able to replicate or divide autonomously once introduced in the host cell, such as bacterial vectors with a bacterial replication origin, or mammalian episomal vectors. Other vectors can become integrated within the host cell genome and thus replicate along with the cell genome.

In a preferred approach to the first aspect of the invention, the replication origins of the DNA of bacteriophage φ29 corresponding to the nucleotide sequences of (a) and (b) are of the same extremity of the DNA of bacteriophage φ29. The DNA extremity of bacteriophage φ29 is preferably the left extremity. The DNA extremity of bacteriophage φ29 is preferably the right extremity.

In a preferred approach to the first aspect of the invention, the replication origin of the DNA of bacteriophage φ29 corresponding to the nucleotide sequence (a) and the replication origin of the DNA of bacteriophage φ29 corresponding to the nucleotide sequence (c) are of different extremities of the DNA of bacteriophage φ29. Preferably, one replication origin of the DNA of bacteriophage φ29 is the left extremity, while the other replication origin of the DNA of bacteriophage φ29 is the right extremity.

In a preferred approach to the first aspect of the invention, the nucleotide sequence comprising the left replication origin of the DNA of bacteriophage φ29 contains between 65 and 200 nucleotides of the left extremity of the DNA of bacteriophage φ29. It preferably contains between 65 and 72 nucleotides of the left extremity of the DNA of bacteriophage φ29.

In a preferred approach to the first aspect of the invention, the nucleotide sequence comprising the right replication origin of the DNA of bacteriophage φ29 contains between 125 and 250 nucleotides of the right extremity of the DNA of bacteriophage φ29. It preferably contains between 150 and 200 nucleotides of the right extremity of the DNA of bacteriophage φ29.

In a preferred approach to the first aspect of the invention, the genic construct moreover includes at least one multiple cloning site (MCS).

The term multiple cloning site (MCS) refers to a small nucleotide sequence containing the target sequences of numerous restriction enzymes.

In a preferred approach to the first aspect of the invention, the genic construct moreover includes at least one marker. The marker preferably is a gene encoding for antibiotic resistance. The antibiotic in question is preferably ampicillin or kanamycin.

The term “marker”, as it is used in this study, refers to a nucleotide sequence that encodes for a marker peptide or marker protein, allowing us to confirm that the vector has been transfected or transduced correctly, and that its sequences are correctly expressed. The marker may be a nucleotide sequence encoding for a fluorescent protein or a gene encoding for antibiotic resistance, used to select the cells that carry the vector.

In a preferred approach to the first aspect of the invention, the cleavage site is the cleavage target nucleotide sequence of the restriction enzyme Dra I or Bae I.

In a preferred approach to the first aspect of the invention, the genic construct comprises SEQ ID NO: 1, SEQ ID NO: 2, SEQ ID NO: 3 or a biologically equivalent variant.

A biologically equivalent variant of a nucleotide sequence is a nucleotide sequence possessing the same biological activity, i.e., comprising the same elements or equivalent elements with the same function.

A second aspect of this invention refers to utilization of the genic construct of the first aspect or approach to the invention for amplifying an exogenous nucleotide sequence.

It is important to distinguish between the replication of a nucleotide sequence and the amplification of a nucleotide sequence. In effect, while replication is a linear process, amplification is an exponential process; consequently, the number of DNA molecules obtained with an amplification reaction is far greater than that obtained with a replication reaction.

The authors of the present invention have demonstrated that it is possible to achieve efficient in vitro amplification by means of the DNA replication machinery of bacteriophage φ29, using as template a linear DNA with minimum replication origins of the DNA of φ29, but without TP covalently bound to the extremities. The DNA inserted between the origins of the DNA of φ29 can be heterologous. The minimum requirements of the in vitro system, as regards protein and DNA sequences, corresponding to the minimum origin of 68 bp of the left extremity of φ29, are shown in FIG. 11. As described by Gutiérrez et al. (Nucleic Acids Research 1988. Vol. 16 (13); 5895-5914), the last 12 bp of the extremities of the DNA of φ29 are needed to start replication, and for prolongation. This 12 bp DNA sequence is active as replication origin in the presence of TP and DNA polymerase, without the presence of any other protein; thus, mostly if not entirely, recognition of the origin must take place by means of the heterodimer TP-DNA polymerase. Although this complex is able to initiate replication with low efficiency, replication is greatly stimulated by the presence of p6. The minimum 68 bp origin of φ29 contains a high-affinity p6 binding site between nucleotides 35 and 68 with respect to the extremity. This nucleation site is necessary and sufficient for securing amplification stimulation mediated by this protein. Another essential protein for DNA amplification is SSB, which shows affinity for single strand DNA, without sequence specificity.

In a third aspect, this invention refers to a oligonucleotide comprising a nucleotide sequence of between 65 and 130 nucleotides from the left extremity of the DNA of bacteriophage φ29. It preferably contains between 67 and 72 nucleotides of the left extremity of the DNA of bacteriophage φ29. The 5′ extremity of the oligonucleotide of this third aspect of the invention is preferably phosphorylated.

In a preferred approach to the third aspect of the invention, the oligonucleotide comprises the target nucleotide sequence of a restriction enzyme or the nucleotide sequence resulting from cleavage with a restriction enzyme in position 3′ with respect to the nucleotide sequence including between 65 and 130 nucleotides or between 67 and 72 nucleotides of the left extremity of the DNA of bacteriophage φ29.

A fourth aspect of the present invention refers to utilization of the oligonucleotide of the third aspect of the invention for constructing a recombinant linear nucleotide sequence in which an exogenous nucleotide sequence destined for amplification is flanked by nucleotide sequences comprising the sequence including between 65 and 130 nucleotides or between 67 and 72 nucleotides of the left extremity of the DNA of bacteriophage φ29.

The authors of this invention have seen that the phosphate group at the 5′ extremities of the replication origins is also essential for DNA amplification, especially for the elongation process. The addition of a small molecule, such as a propyl group, to the 5′ phosphate impedes use of the origin for amplification.

A fifth aspect of the present invention refers to a DNA amplification method comprising at least the following steps:
a) obtainment of a linear DNA molecule comprising the DNA sequence destined for amplification and flanked on both extremities by:
   i) the sequence of the left replication origin of the DNA of bacteriophage φ29, the terminal extremity of this origin being located in the linear DNA extremity; or by
   ii) the sequence of the right replication origin of the DNA of bacteriophage φ29, the terminal extremity of this origin being located in the linear DNA extremity; or by
   iii) the sequence of the right replication origin of the DNA of bacteriophage φ29 on one side and of the left replication origin on the other—the terminal extremity of these origins being located in the two extremities of the linear DNA;
b) amplification of the linear DNA sequence obtained in step (a).

In a preferred approach to the fifth aspect of the invention, use is made of a DNA polymerase, a terminal protein (TP), a single strand DNA binding protein (SSB) and a double strand DNA binding protein (DBP) in the amplification of step (b). The proteins DNA polymerase, TP, p5 and p6 of bacteriophage φ29, or any bioequivalent variant of these proteins, are preferably used.

A bioequivalent variant of a protein or a biologically equivalent variant of a protein is a protein possessing the same biological activity, i.e., with the same function. In the present example, this may be DNA polymerase activity, primer activity in the case of TP, double strand DNA binding in the case of p6, or single strand DNA binding in the case of protein p5.

In a preferred approach to the fifth aspect of the invention, the DNA sequence destined for amplification is between 500 and 100,000 bp in size.

In a preferred approach to the fifth aspect of the invention, the linear DNA sequence obtained in step (a) presents phosphorylation of both 5′ extremities.

In a preferred approach to the fifth aspect of the invention, the sequence of the left replication origin of the DNA of bacteriophage φ29 contains between 65 and 200 nucleotides of the left extremity of the DNA of bacteriophage φ29. It preferably contains between 67 and 72 nucleotides of the left extremity of the DNA of bacteriophage φ29.

In a preferred approach to the fifth aspect of the invention, the sequence of the right replication origin of the DNA of bacteriophage φ29 contains between 125 and 250 nucleotides of the right extremity of the DNA of bacteriophage φ29. It preferably contains between 150 and 200 nucleotides of the right extremity of the DNA of bacteriophage φ29.

In a preferred approach to the fifth aspect of the invention, the sequence of the left replication origin of the DNA of bacteriophage φ29 contains between 65 and 200 nucleotides or between 67 and 72 nucleotides of the left extremity of the DNA of bacteriophage φ29, and the sequence of the right replication origin of the DNA of bacteriophage φ29 contains between 125 and 250 nucleotides or between 150 and 200 nucleotides of the right extremity of the DNA of bacteriophage φ29.

In a preferred approach to the fifth aspect of the invention, step (b) is carried out at a temperature of under 30° C., preferably under 27° C., and even more preferably at a temperature of between 20-25° C.

In a preferred approach to the fifth aspect of the invention, step (a) makes use of the genic construct of the first aspect of the invention. In a preferred approach to the fifth aspect of the invention, step (a) makes use of the oligonucleotide of the third aspect of the invention.

A sixth aspect of the present invention refers to a kit for the amplification of an exogenous nucleotide sequence, consisting of at least a genic construct according to the first aspect of the invention, at least an oligonucleotide according to the third aspect of the invention, or both.

In a preferred approach to the sixth aspect of the invention, the genic construct comprises SEQ ID NO: 1, SEQ ID NO: 2 or SEQ ID NO: 3.

In a preferred approach to the sixth aspect of the invention, the kit moreover includes a DNA polymerase, a terminal protein (TP), a single strand DNA binding protein (SSB) and/or a double strand DNA binding protein (DBP). The DNA polymerase, the TP, the single strand DNA binding protein (SSB) and/or the double strand DNA binding protein (DBP) are preferably the DNA polymerase, TP, p5 and/or p6 of bacteriophage φ29, or a bioequivalent variant of these proteins.

In a preferred approach to the sixth aspect of the invention, the kit moreover comprises at least one of the elements of the following list: deoxynucleotide triphosphates (dNTPs), (NH₄)₂SO₄, MgCl₂, dithiothreitol (DTT), glycerol, bovine serum albumin (BSA) and Tris-HCl buffer with a pH of between 6.5 and 8.

A seventh aspect of this invention refers to utilization of the kit of the sixth aspect of the invention for amplifying an exogenous nucleotide sequence.

Throughout the description and the claims, the term “comprises” and its variants do not aim to exclude other technical characteristics, additives, components or steps. For the experts in the field, other objects, advantages and characteristics of the invention will be drawn in part from the description, and in part from implementation of the invention. The following examples are provided for illustrative purposes, and do not aim to impose limitations upon the present invention.

### DESCRIPTION OF THE FIGURES

FIG. 1. Schematic representation of the terminal protein (TP)— primed replication of the DNA of bacteriophage φ29. The initiator and paternal TP are shown in black and gray, respectively. The different replication phases are indicated to the right of the figure.

FIG. 2. Genic construct presenting the replication origins of the DNA of bacteriophage φ29. A. The pETORPHI plasmid was constructed by cloning a DNA fragment synthesized by Genescript, containing fused fragments of 194 base pairs (bp) and 191 bp from the left and right extremities of the genome of bacteriophage φ29, respectively, between sites Sac I and Hind III of pET28b. B. The extremities were inverted with an “extremity-to-extremity” orientation, generating a Dra I site at the joining point, as shown in the figure. “Ori L” and “Ori R” indicate the left and right origins of the DNA of φ29, respectively. C. The DNA fragment produced by plasmid linearization with Dra I, which presents the extremities of the DNA of φ29 correctly oriented. MCS1 and MCS 2 indicate the multiple cloning sites 1 and 2.

FIG. 3. The amplification of pETORPHI is more effective at 22° C. The DNA bands in agarose gel obtained in the amplification assays are shown. The amounts in nanograms (ng) of pETORPHI plasmid or TP-DNA templates of bacteriophage φ29 used in the amplification assays are shown. Both the incubation temperatures and the absence or presence of p6 are indicated. The reactions incubated at 30° C. contained p6 in all cases. Use was made of 37.5 ng of TP and 25 ng of DNA polymerase. The size of the amplified bands is indicated: 19 kilobases (kb) for φ29 TP-DNA and 5.8 kb for pETORPHI cleaved with Dra I. The amplification factors calculated at 22° C. were 80-fold for TP-DNA and 24-fold for pETORPHI.

FIG. 4. Amplification requires two extremities of φ29 DNA in the same molecule. The pETORPHI plasmid was cleaved with the enzymes shown in the figure. A. Fragments resulting from the digestion of pETORPHI with the enzymes Dra I and Mlu I or Pvu I. B. DNA bands in agarose gel resulting from the amplification reaction using φ29 TP-DNA or the different plasmid fragments as template. The amounts (in ng) of φ29 TP-DNA or of fragments of the pETORPHI plasmid are shown. The bands corresponding to the plasmid cleaved with Mlu I or Pvu I, in addition to Dra I, are 4.6 and 4.4 kb in size, respectively. The amplification factors 109-fold for TP-DNA and 40-fold for pETORPHI.

FIG. 5. The amplification kinetics of pETORPHI are similar to those of the genome of bacteriophage φ29. A. Products of the amplification reactions in agarose gel. These reactions contained 25 ng of φ29 TP-DNA or pETORPHI cleaved with Dra I; incubation was carried out at 22° C. during the specified periods of time, and the reactions were stopped by adding 0.1% SDS and EDTA 10 mM. The amounts of TP and DNA polymerase were 37.5 and 25 ng, respectively. B.

The gel bands of the experiments described in A were quantified using the Fosforimager system, and the values obtained were represented as a function of time. The absolute values are shown in section B. C. Representation of the values quantified in B, standardized with respect to the value at one hour (h). The amplification factors were 32-fold for TP-DNA and 14-fold for pETORPHI.

FIG. 6. The minimum amount of template needed for amplification is seen to be 5 ng. The amplification reactions were established with the indicated amounts (in ng) of φ29 TP-DNA or pETORPHI plasmid cleaved with Dra I, and incubated at 22° C. during two hours. The amplification factors for 5 ng were 309-fold for TP-DNA and 141-fold for pETORPHI.

FIG. 7. A 68 bp fragment from the left extremity of the DNA of bacteriophage φ29 is seen to be an efficient amplification origin. A. The pETORPHI68L plasmid was constructed by cleaving pETORPHI with Dra I and EcoR Ito eliminate the 191 bp fragment, corresponding to the right extremity of the genome of φ29, with replacement by a 68 bp fragment from the left extremity of the genome of φ29 (shown at left). B. For the amplification reaction, pETORPHI68L was cleaved with Dra I, and the reaction was incubated at 22° C. with 25 ng of the corresponding template. Numbers 1 and 2 of the lanes with pETORPHI68L correspond to two different clones of this plasmid. The negative control in the absence of template is indicated as −. TP-DNA and pETORPHI were used as positive controls.

FIG. 8. Ligation of a 68 bp DNA fragment from the left extremity of the genome of bacteriophage φ29 to the two extremities of a heterologous DNA fragment allows amplification of the latter. A. DNA fragments corresponding to the 68 bp origin of the left extremity of the genome of φ29 were assembled, using single strand oligonucleotides that generated cohesive extremities for enzymes BsmB I (indicated as 68L) and EcoO109 I (indicated as 68L′). The pEYFP—N1Bsm plasmid was cleaved with BsmB I and EcoO109 I, and the fragments of this plasmid were included in a ligation reaction with the described oligonucleotides 68L and 68L′. B. Amplification requires the presence of the extremities of the genome of φ29 at both extremities of the heterologous DNA, since amplification is only observed when the template is the product of ligation of the heterologous DNA with nucleotides 68L and 68L′. Ligation was carried out either with oligonucleotides 68L and 68L′ and fragment 1 of the pEYFP-N1 Bsm plasmid (lanes 1 and 2 indicated as 68L-DNA-68L′) or with only one of the nucleotides plus fragment 1 (lanes 1 and 2 indicated as 68L-DNA), and also in the absence of fragment 1 and with the two types of nucleotides, as control (lanes 1 and 2 indicated as 68L and 68L′). In the amplification reactions, aliquots of the ligations were used as templates; accordingly, each reaction contained the same number of moles for both the fragment pertaining to the plasmid (4 kb) and for pETORPHI.

FIG. 9. The phosphate group in position '5 is necessary for amplification. A. The DNA bands obtained in the amplification reactions of aliquots of pETORPHI treated with Antarctic phosphatase (+Pase), with adenosine triphosphate (ATP) and T4 polynucleotide kinase, after phosphatase (+Pase +PNK), or subjected to the same processing as the treated samples, but without adding enzymes (pETORPHI*). In all cases, the amount of pETORPHI present in the reactions was 25 ng. A reaction in the absence of DNA template was used as negative control. B. The same ligations as those described in FIG. 8B were performed. In addition, the oligonucleotides were designed to present no modification at their 5′ extremities 5′ (−), phosphorylation at 5′ (P), or propyl group bound to the phosphate at 5′ (Prop), or unmodified oligonucleotides were treated with ATP and T4 polynucleotide kinase after ligation (+PNK). Lanes 1 indicate that only oligonucleotide 68L was used in ligation, and lanes 2 indicate that both oligonucleotides (68L and 68L′) were used. C. Schematic representation of the chemical structures located at extremity 5′ of the oligonucleotides: unmodified (−), modified with a phosphate group (P), modified with a propyl group bound to the phosphate (Prop), and bound to the serine group of TP, through the phosphate group.

FIG. 10. A pETORPHI derivative called pETORPHIBae, linearized with Bae I is seen to be effective as a template for amplification. A. Schematic representation of the recognition site of Bae I (underlined) and of the cleaving points (arrows) and positions of the origins of φ29 (capital letters) in pETORPHIBae. B. The DNA bands resulting from the amplification reactions using TP-DNA and pETORPHI as positive controls, the absence of DNA as negative control (−), and the pETORPHIBae plasmid linearized with Bae I without modifications, and with different treatments indicated as “Pretrat” and “Klenow”. “Pretrat” indicates that pETORPHIBae linearized with Bae I was pretreated for 5 minutes (min) with the DNA polymerase of φ29, after which the rest of the components of the reaction were added, except TP, which was added 5 min later. “Klenow” indicates that pETORPHIBae linearized with Bae I was treated for 10 min with the Klenow enzyme in the presence of deoxyribonucleoside triphosphates (dNTPs); the mentioned enzyme was then deactivated, and the rest of the components of the reaction were added. Aliquots of 25 ng of pETORPHIBae linearized with Bae I were used in the three amplification reactions.

FIG. 11. The requirements of the origins of the DNA of bacteriophage φ29 for in vitro amplification. The origin of the 68 bp left extremity of φ29 DNA is shown. The 12 bp located on the same extremity (rectangle) are necessary for the initiation and elongation reactions performed by the DNA polymerase of φ29, forming a heterodimer composed of DNA polymerase (represented as an oval) bound to TP as initiator (Init. represented in the form of a rectangle). The phosphate group, located at extremity 5′ (P), was seen to be essential for in vitro amplification. Protein p6 (hexagon) and the high affinity sequences (rectangle between positions 35 bp and 68 bp) are necessary for stimulating initiation by this protein. Amplification requires SSB (rhombus) to bind to the displaced DNA strand, represented as a broken line.

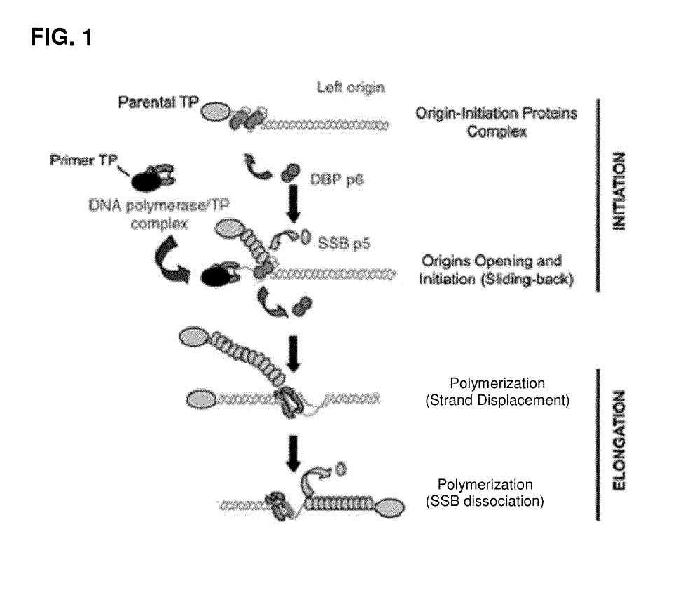

### EXAMPLES

The invention is illustrated below by means of a series of assays made by the inventors, demonstrating the efficacy of the amplification method of the invention, as well as of the nucleotide sequences used to develop the technique.

### Example 1

### Design of a Plasmid Generating the Functional Extremities of the DNA of Bacteriophage φ29 when Linearized

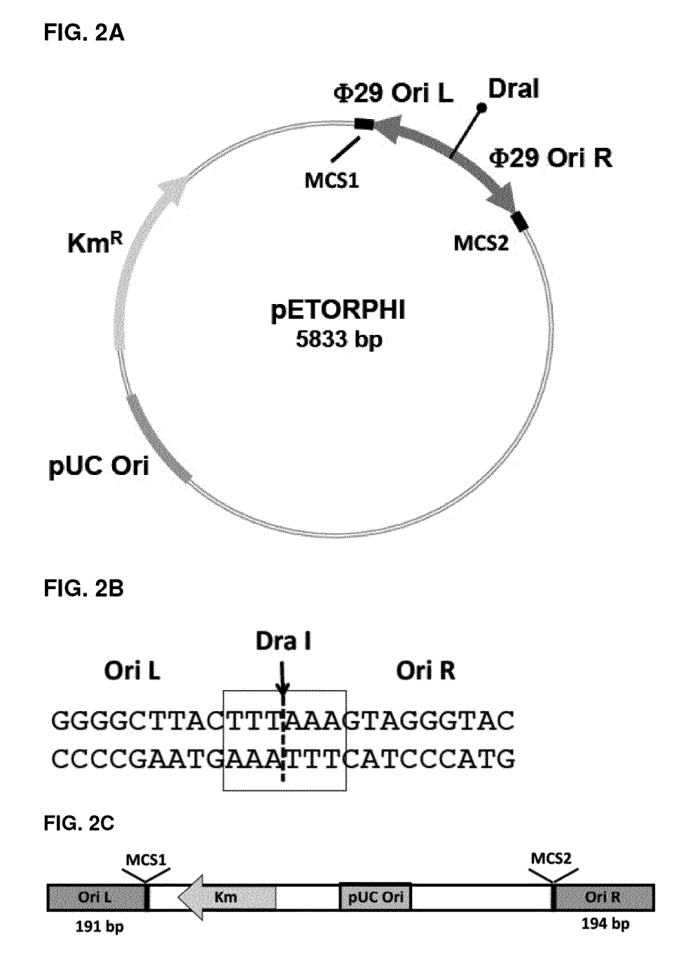

We designed the plasmid described in FIG. 2A, called pETORPHI (SEQ ID NO: 1). The pETORPHI plasmid was constructed by cloning between sites Sac I and Hind III of pET28b (SEQ ID NO: 11) a DNA fragment synthesized by Genescript, containing fused fragments of 194 base pairs (bp) and 191 bp from the left and right extremities of the genome of bacteriophage φ29, respectively. The extremities were inverted with an “extremity-to-extremity” orientation, generating a single Dra I restriction site at the joining point, as shown in FIG. 2B. The pETORPHI possesses a backbone that contains no Dra I restriction site, a multiple cloning site (MCS), a pUC type replication origin (Ori), a resistance marker consisting of a kanamycin resistance gene, and the right and left extremity sequences of the DNA of the genome of bacteriophage φ29, reaching base pairs (bp) 191 and 194 from the extremity, respectively (SEQ ID NO: 12 and SEQ ID NO. 13, respectively). After cleaving the plasmid with Dra I, the two extremities of the linearized plasmid are identical to the two extremities of the DNA of φ29 up to the aforementioned positions (FIG. 2C).

### Example 2

### Amplification Using the Linearized pETORPHI Plasmid as Template

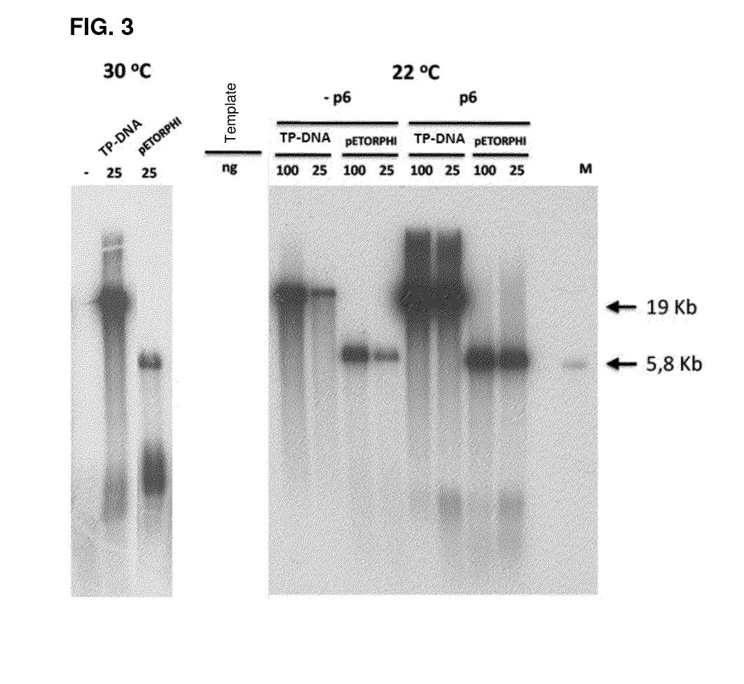

We checked the capacity of the pETORPHI plasmid cleaved with Dra I (henceforth referred to as pETORPHI) to act as a template in amplification started with terminal protein (TP) by means of the proteins DNA polymerase, p5 and p6 of bacteriophage φ29. As can be seen in FIG. 3, at left, on incubating the amplification reaction at 30° C. during two hours and using the genome of φ29 with TP bound to its 5′ extremities, we obtain an amplified product in the expected position. Lesser amounts of the correct product (5.8 kb) are obtained on using pETORPHI as template, and a larger amount of material of lesser molecular weight is produced. Surprisingly, when the amplification reaction is carried out at 22° C., the presence of these products of lesser molecular weight was found to decrease. In the absence of p6 there is a certain amount of correct amplified products, though the reaction does not reach maximum performance—not even starting with 100 ng of genome of bacteriophage φ29 or plasmid as template. In the presence of p6, the reaction reaches a plateau with 25 ng of φ29 genome or with the same amount of pETORPHI, and most of the amplified DNA is moreover in the correct position, with almost no lesser molecular weight products. All the subsequent experiments were made at 22° C. DNA band quantification was carried out with the Fosforimager system. The calculated amplification factors are 80-fold for the bacteriophage genome and 24-fold for the pETORPHI plasmid.

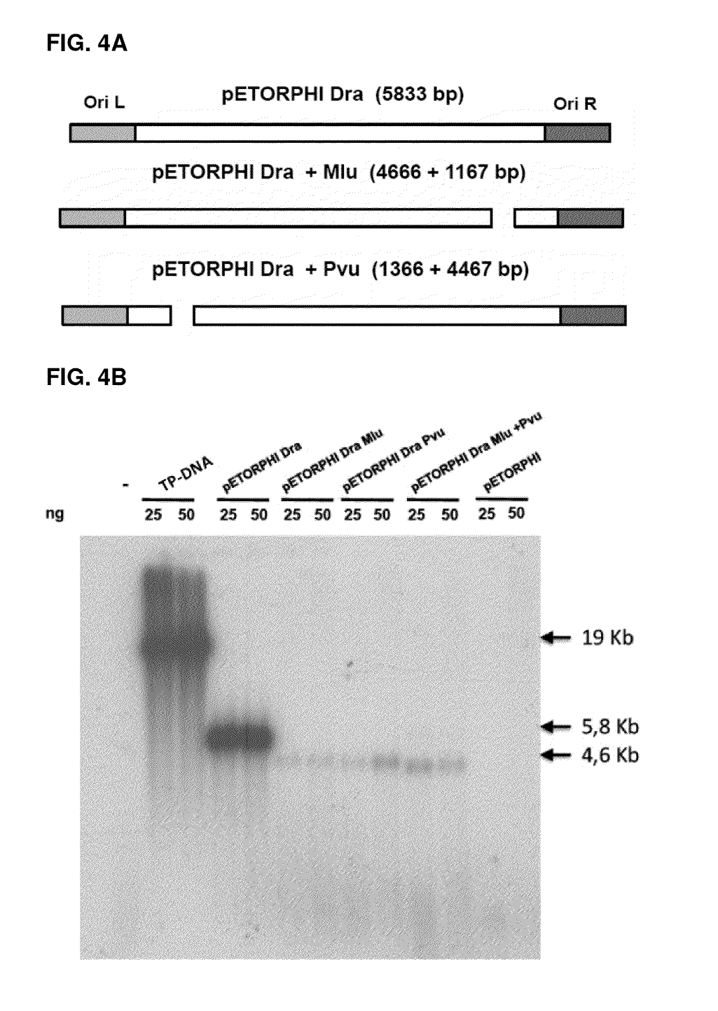

In order to determine whether the products were amplification products or linear replication products, the origins of the DNA of φ29 of the pETORPHI plasmid were separated into two molecules, so that both DNA fragments would not be susceptible to exponential amplification, since each DNA molecule would only contain one of the φ29 replication origins—thereby giving rise to linear replication. We chose two enzymes, Mlu I and Pvu I, which each cleave pETORPHI once, giving rise to a series of fragments of about 4.7 and 1.2 Kb, and 1.4 and 4.5 Kb in size, respectively, on using these enzymes with Dra I, as can be seen in FIG. 4A. Aliquots of the linearized pETORPHI plasmid were treated using Dra I, with either Mlu I or Pvu I, or with restriction buffer as control, using them as templates in the amplification reaction. FIG. 4B shows that in all cases in which we separate the two extremities of the pETORPHI plasmid, the amount of amplified DNA decreases drastically (about 30-fold) when compared with the signal obtained using pETORPHI. Even when the fragments are cleaved with the two enzymes independently and are mixed in the amplification reaction to check whether they hybridize or complement each other (FIG. 4A, lanes Dra Mlu+Pvu), the results are almost identical to those obtained with fragments cleaved with only one enzyme. The non-cleaved pETORPHI plasmid produces no signal. Likewise, it was checked that the different templates had active origins referred to the start of replication, to ensure that the amplification defects are only due to separation of the two origins into two molecules.

The results indicate that the pETORPHI plasmid gives rise to true amplification, using the origins located at both extremities of the molecule.

### Example 3

### Amplification Kinetics

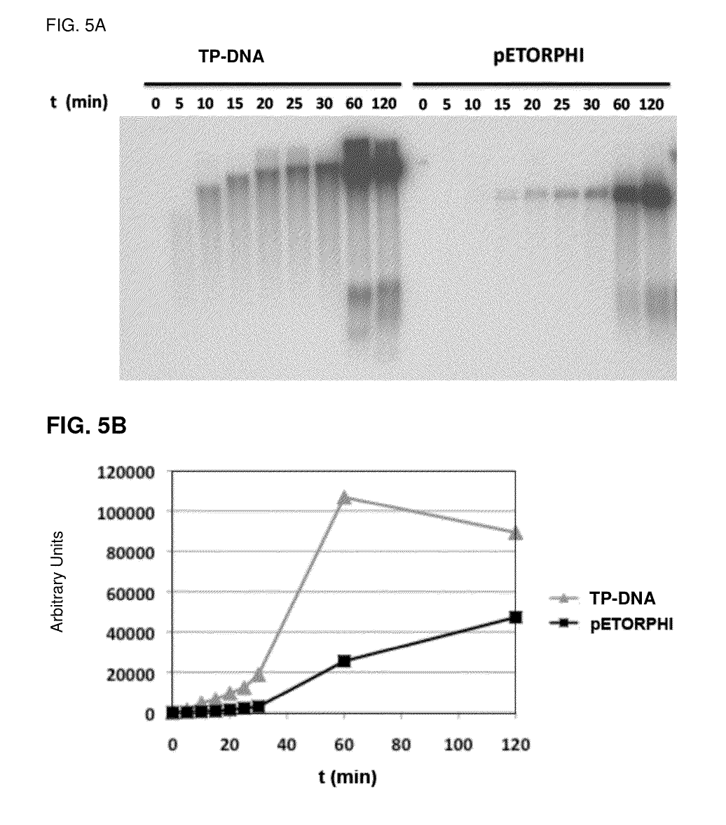

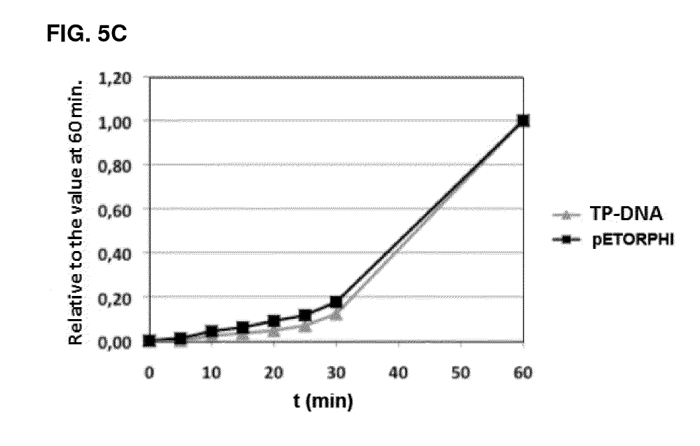

Amplification experiments were carried out, detecting the products over time and comparing the results obtained using the genome of bacteriophage φ29 or pETORPHI as template. FIG. 5A shows that both templates yield a very similar profile, with small amounts of products generated up to 30 minutes (min), followed by a gradual increase to a maximum reached after 60 min with the genome of φ29, or one-half of the maximum with pETORPHI. Having reached this point, pETORPHI continues to generate products for another 60 min. Quantification using the Fosforimager system showed the amount of synthesized TP-DNA to increase up to 60 min, where the TP-DNA amplification saturation point is reached (FIG. 5B). In the case of the pETORPHI vector, amplification continues until saturation is reached after 120 min. The values obtained up to 60 min, standardized by taking the value at 60 min as equal to 1, are practically identical for the genome of φ29 and pETORPHI (FIG. 5C). This indicates that up until 60 min, the amplification reaction proceeds in a very similar manner for both TP-DNA of φ29 and pETORPHI, with the difference that saturation has already been reached after 60 min with TP-DNA, while pETORPHI continues to generate amplification product for an additional hour. Longer incubation times of up to 5 hours (h) did not imply an increase in total product generated with either TP-DNA of φ29 or pETORPHI.

### Example 4

### Minimum Amount of Template Required for Amplification

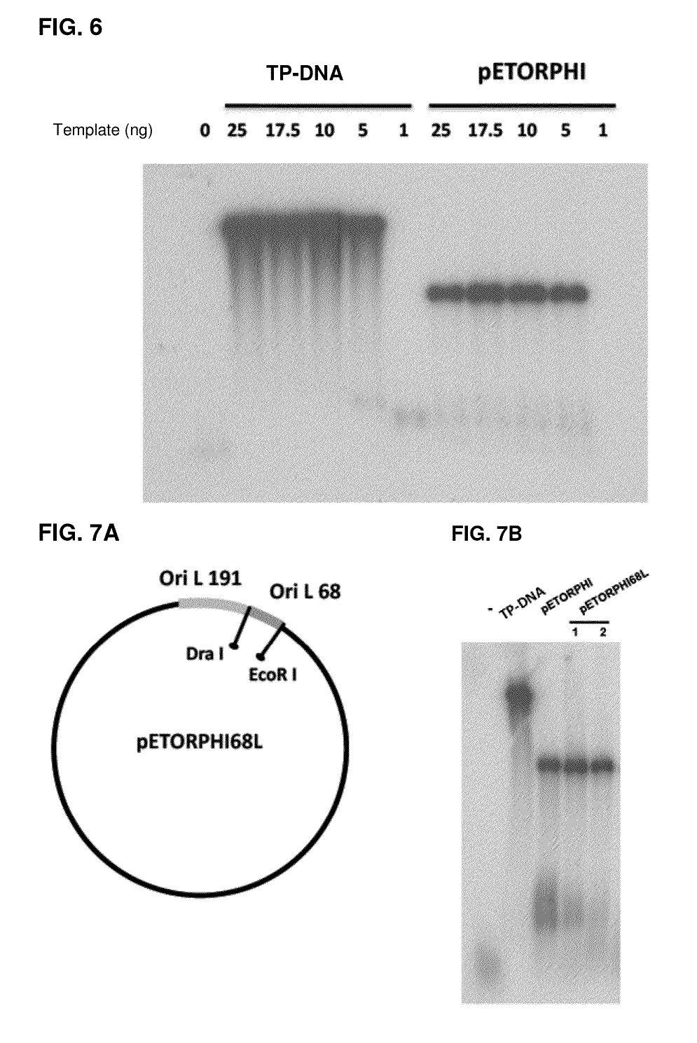

We determined the minimum amount of template required for amplification. FIG. 6 shows that on using the genome of φ29 as template, the minimum amount allowing amplification is 5 ng, and the amount of product obtained is very similar when using 10 to 25 ng of template. This indicates that amplification is close to the saturation point in that template concentration interval. No amplification is obtained with 1 ng. The results obtained on using pETORPHI as template are similar. In this case 3.3 times more template molecules were used than with the genome of φ29. It is important to point out that on starting the reaction, pETORPHI does not have the paternal TP, while the genome of φ29 does have the paternal TP, and the latter increases the affinity of the extremities with respect to the replication machinery.

### Example 5

### Minimum Replication Origin

The minimum length of DNA of bacteriophage φ29 allowing replication activity stimulated with p6 corresponds to the last 68 bp of the left extremity of the genome of φ29. This includes the inverted repetition of 6 bp (5′ AAAGTA 3′) and the sequences of 35 to 68 bp of the left extremity that constitute high affinity sites for the binding of p6.

The 194 bp segment corresponding to the right extremity of the genome of φ29 of pETORPHI was replaced by the 68 bp minimum replication origin of the left extremity, described above, to obtain the so-called pETORPHI68L (SEQ ID NO: 2) (FIG. 7A).

The 68 bp fragment was cloned with an inverted orientation, regenerating the Dra I restriction site as in pETORPHI, in order to obtain the DNA extremities of φ29 when pETORPHI68L is linearized with Dra I. FIG. 7B shows that the capacity of pETORPHI68L to act as an amplification template is the same as that of the original pETORPHI.

### Example 6

### Amplification after Ligation with Oligonucleotides that Include the Minimum Origins of the DNA of φ29

After confirming that a 68 bp fragment of the left extremity of the DNA of bacteriophage φ29 is an effective amplification origin, we can design oligonucleotides which following hybridization generate the 68 bp fragment as double strand DNA to which we can add the sequence of interest, such as for example single strand DNA extensions to ligate them to extremities of exogenous DNA obtained after digestion with restriction enzymes. By means of this process we can generate oligonucleotides of φ29 DNA with which the minimum replication origin of φ29 can bind to any linear DNA chosen for amplification.

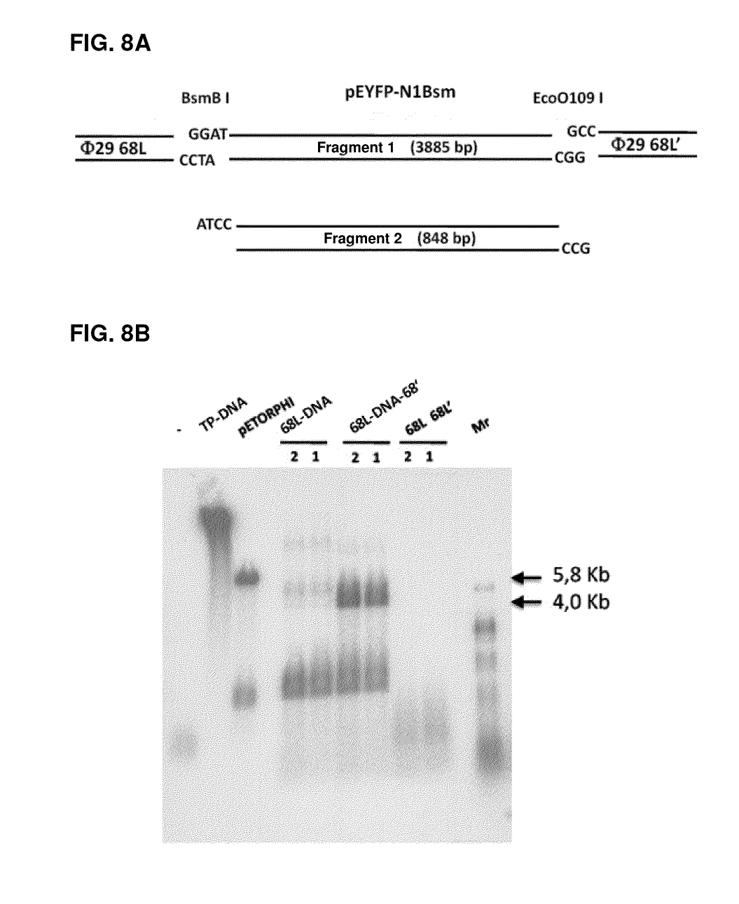

Using guided mutagenesis, we introduced the sequence of the target of restriction enzyme BsmB I in the pEYFP-N1 plasmid (SEQ ID NO: 14) (Clontech) in position 4702 to generate the pEYFP-N1Bsm plasmid (SEQ ID NO: 15). Posteriorly, pEYFP—N1Bsm was cleaved with the enzymes BsmB I and EcoO109 I, which recognize single sequences in that vector. Likewise, we used two pairs of synthetic oligonucleotides (SEQ ID NO: 7 and SEQ ID NO: 8), whereby after hybridization, each pair would form the 68 bp double strand DNA corresponding to the minimum origin of φ29, in addition to short single strand extensions allowing ligation to one or other of the extremities obtained on cleaving the pEYFP—N1 Bsm plasmid with enzyme BsmBI or EcoO109I, respectively (FIG. 8A). We chose these enzymes because they generate asymmetrical extremities, thereby avoiding the auto-ligation of oligonucleotides that contain the same cohesive extremity, as well as cross-ligation between sites BsmB I and EcoO109 I. In this way, following hybridization, each pair of oligonucleotides can ligate to the complementary restriction extremity of the plasmid, but not to the other extremity, or to the other pair of oligonucleotides, and likewise cannot undergo auto-ligation. On the other hand, this restriction based approach makes it unnecessary to carry out gel purification of the DNA fragments or ligation products. Following the ligation reaction, the ligase was inactivated and the sample was treated with adenosine triphosphate (ATP) and T4 polynucleotide kinase.

Utilization of the DNA resulting from the ligation reactions as templates only allows real amplification of one of the ligation products, namely the 3885 bp fragment of the pEYFP-N1Bsm plasmid (FIG. 8A, fragment 1), ligated to the two oligonucleotides with sequences that comprise the 68 bp of the left extremity of the DNA of bacteriophage φ29. This product generates a single 4021 bp band corresponding to the scheme: φ29 68L-BsmBI-pEYFP-N1 Fragment 1-EcoO109I-φ29 68L.

The other fragment (FIG. 8A, fragment 2) does not ligate to the oligonucleotides, and is therefore not amplified. As can be seen in FIG. 8B, only ligation comprising all the adequate components generates an amplified band of the appropriate length, while the control reactions performed with ligation mixture in the absence of oligonucleotides (68L-DNA), or containing only the two oligonucleotides (68L and 68L′, without plasmid fragment), did not produce the correct amplified product. After quantification with the Fosforimager system, we calculated that the amplified band obtained with the ligation product corresponded to 50% of the molecules present in the pETORPHI band. The results confirm that it is possible to ligate minimum origins of DNA of bacteriophage φ29 to heterologous DNA to obtain TP-DNA of adequate length by means of the replication system of φ29, without the need for gel purification or cloning.

### Example 7

### The Phosphate Group at the 5′ Extremities of the DNA is Necessary for Amplification

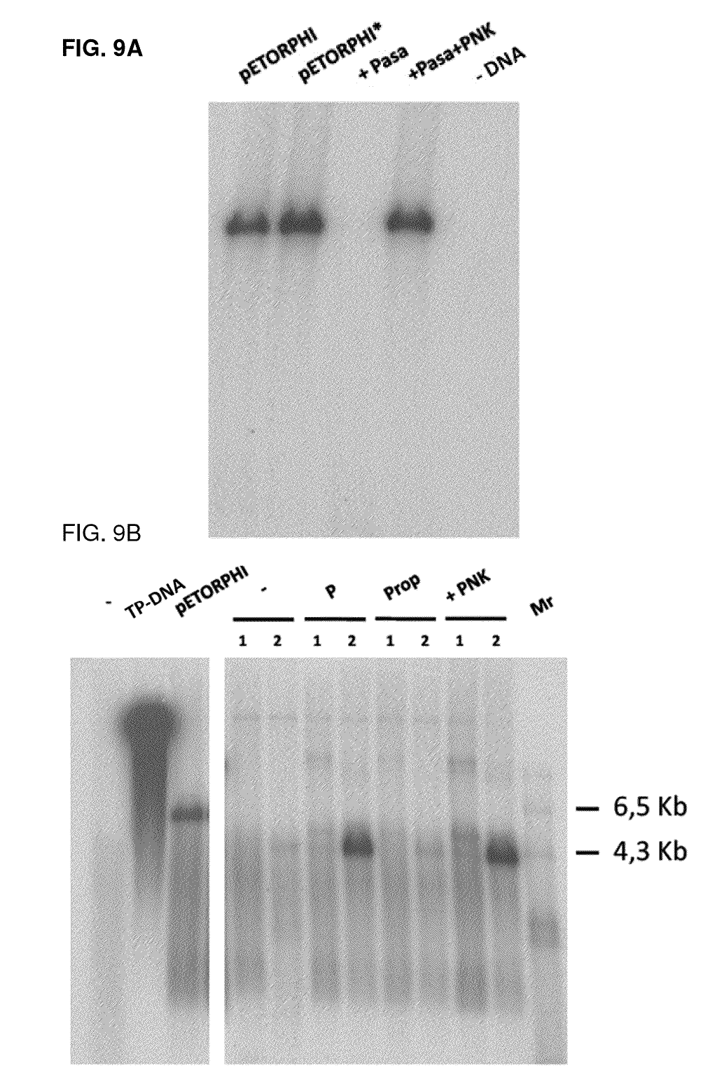

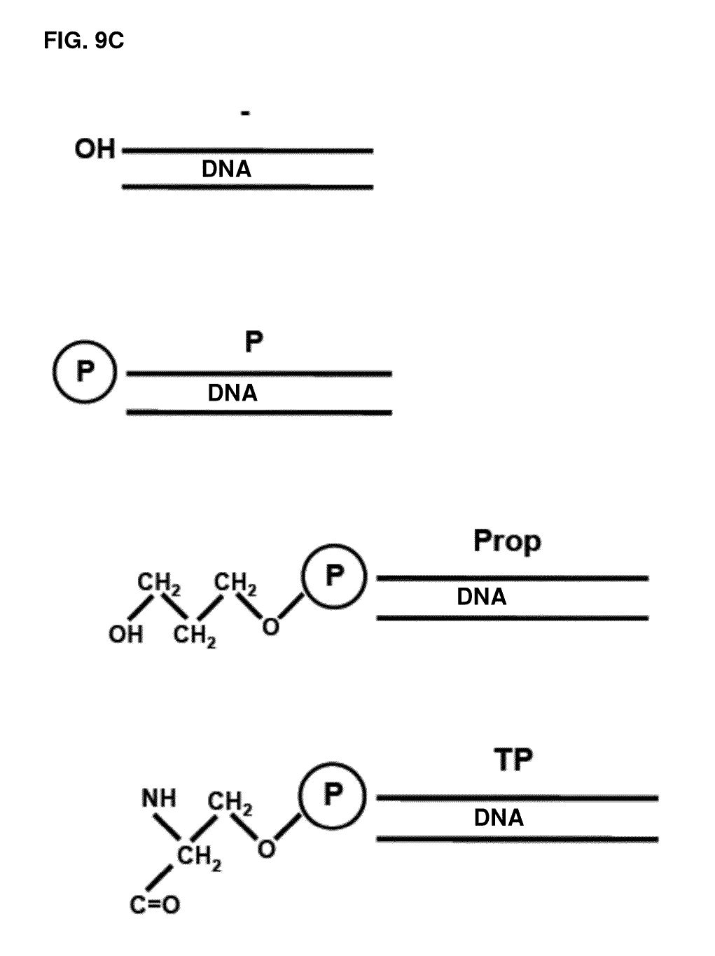

As described above, during the experiments carried out with oligonucleotides containing the minimum origins of φ29, we found that the extremities of the DNA of bacteriophage φ29 containing non-phosphorylated oligonucleotides at the 5′ extremity could not be used as amplification template. Therefore, we treated the ligation products with ATP and T4 polynucleotide kinase, after which amplification of the templates obtained by ligation of the abovementioned oligonucleotides was effectively observed. In the above section, in order to avoid ligation of the extremities of the DNA of φ29 with themselves through their blunt extremities, the oligonucleotides which we used were previously dephosphorylated and, after ligation, were treated with ATP and T4 polynucleotide kinase. In order to confirm the importance of the 5′ phosphate group in the amplification process, we carried out the experiment shown in FIG. 9A. An aliquot portion of pETORPHI was cleaved with Dra I and treated with Antarctic phosphatase (New England Biolabs). Then, one half of the sample was treated with ATP and T4 polynucleotide kinase, while the other half was treated only with the kinase buffer, as control. After precipitating the samples and subjecting them to the amplification protocol, we found that the sample treated only with phosphatase yielded no amplification product. In contrast, both pETORPHI control and pETORPHI treated with kinase after phosphatase yielded the typical amount of amplified product after using pETORPHI as template (FIG. 9).

In order to explore the selectivity of the replication machinery for the 5′ extremities of the replication origins of φ29, we synthesized three oligonucleotides with the sequence of the 68 bp left origin of φ29 (SEQ ID NO: 5) and different modifications of the 5′ extremity. Of the three oligonucleotides, one was not phosphorylated, another was phosphorylated at the 5′ extremity, and the third received a propyl group added through the 5′ phosphate. This latter oligonucleotide was designed to imitate the chemical structure bound to the 5′ phosphate when TP is covalently bound to the DNA (FIG. 9C). Following hybridization to form the 68 bp left replication origin, these oligonucleotides were ligated to Fragment 1 of the pEYFP—N1 plasmid (FIG. 8A), and we checked the capacity of the resulting products to act as amplification template. The results shown in FIG. 9B again demonstrate that the DNA generated after ligation of the dephosphorylated oligonucleotide is not a good template for amplification (lane −); in contrast, the DNA ligated to the phosphorylated oligonucleotide (lane P) or DNA phosphorylated after ligation (lane+PNK) constitute functional templates. On the other hand, the oligonucleotide synthesized with a propyl group bound to the 5′ phosphate (lane Prop) did not give rise to amplification. These results show that we can use oligonucleotides with phosphorylated replication origins of φ29 for ligation to heterologous DNA, thereby yielding amplification of the global construct. In this context, an important requirement for amplification is to ensure that the 5′ extremities of the origins are phosphorylated.

### Example 8

### Linearization of pETORPHI with Enzyme Bae I

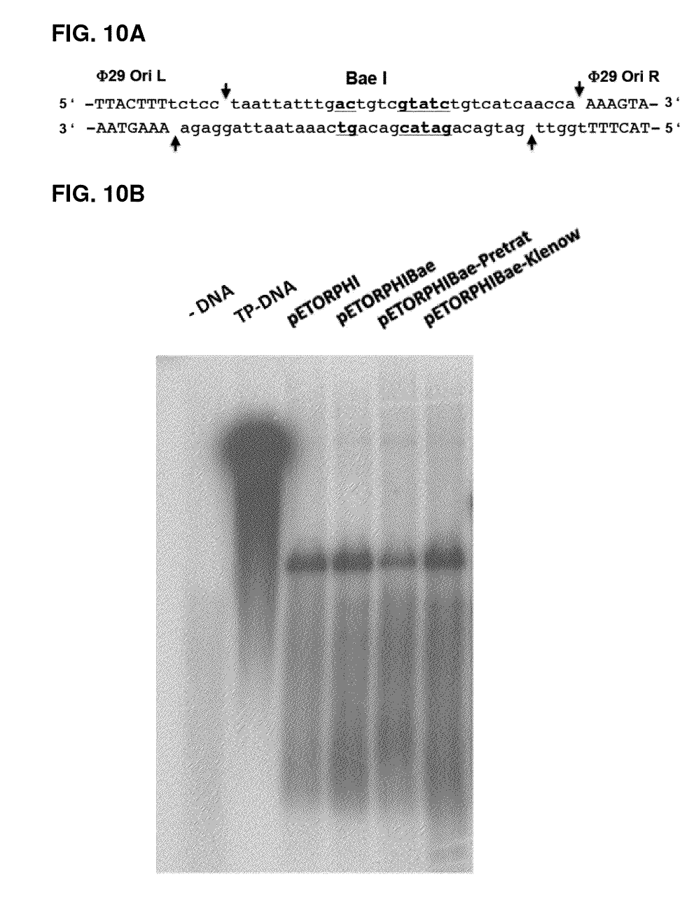

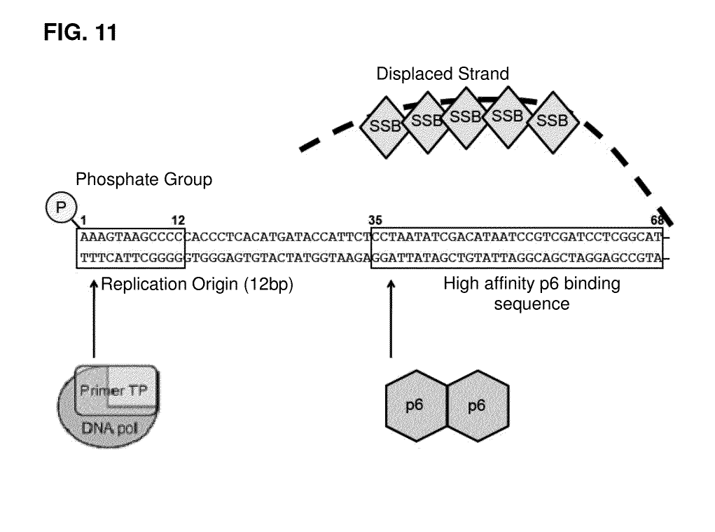

We prepared a pETORPHI derivative called pETORPHIBae (SEQ ID NO: 3), in which a Bae I restriction site was inserted precisely at the junction of the extremities of the DNA of bacteriophage φ29, thereby eliminating the Dra I site. Bae I cleaves double strand DNA at two different points separated by 33 bp; presents its recognition site approximately half way between them; and cleaves DNA independently of the sequence located at the corresponding digestion points. Cleavage with Bae I leaves 5-nucleotide single band extensions at the two resulting 3′ extremities (FIG. 10A). In order to assess the effect of the extensions at the 3′ extremities of the replication origins of φ29 upon amplification efficiency, we performed amplification using three different approaches: (i) amplification reaction under standard conditions after treatment with Bae I (lane pETORPHIBae); (ii) pretreatment of Bae I-cleaved pETORPHIBae with the DNA polymerase of φ29 in the presence of dNTPs, in order to eliminate the 3′ extensions, followed by the addition of TP and the rest of the reaction components; and (iii) pretreatment with Klenow DNA polymerase and nucleotides during 10 min, likewise to produce blunt extremities, followed by addition of the components for carrying out a standard amplification reaction. As can be seen in FIG. 10B, approaches (i) and (iii) give rise to amplification very similar to that obtained with pETORPHI, while pretreatment with the DNA polymerase of φ29 yields poorer results—possibly due to the great activity of the exonuclease of this enzyme.

### Example 9

### Genic Constructs

Genescript synthesized a DNA fragment corresponding to the extremities of the DNA of bacteriophage φ29 up to positions 194 and 191 bp, counting from the left and right extremities, respectively, of the genome of φ29. The fragment was designed with the extremities of the DNA of φ29 fused in an opposite orientation and forming a Dra I restriction site. The fragment was obtained cloned in the pUC57 plasmid (pUC570RPHI) (SEQ ID NO: 4). The pETORPHI plasmid (SEQ ID NO: 1) was constructed by cloning a DNA fragment extracted from pUC570RPHI, using the same restriction enzymes, between sites Sac I and Hind III of pET28b (SEQ ID NO: 11). In this way we obtained a single Dra I site in pETORPHI, and this site was located precisely at the junction between the replication origins of φ29.

In order to construct pETORPHI68L (SEQ ID NO: 2), we designed oligonucleotides which after hybridization conformed the last 68 bp of the left extremity of the DNA of bacteriophage φ29, in addition to an AATT extension to allow ligation to EcoR I sites (SEQ ID NO: 6).

pETORPHI was digested with Dra I and EcoR I in order to eliminate the 191 bp fragment corresponding to the right extremity of the DNA of φ29 from the vector, and the 68 bp fragment of the left extremity of the genome was cloned to replace the eliminated fragment. pETORPHIBae (SEQ ID NO: 3) was created by cleaving pETORPHI with Dra I and inserting a double strand phosphorylated oligonucleotide possessing a recognition site for Bae I (SEQ ID NO: 9 as direct strand and SEQ ID NO: 10 as reverse strand). In this way the cleavage site for that enzyme is located in a position immediately adjacent to the terminal extremities of the origins of φ29 in the 5′ strand, while a 5-nucleotide extremity is left in the 3′ strand (FIG. 10A).

### Example 10

### Enzymatic Digestions, Ligation Reactions and Dephosphorylation

Digestion of the vectors with the enzyme Dra I was carried out normally during four hours, using buffer 4 of New England Biolabs, except in the cases requiring multiple digestions with the enzymes Dra I, Mlu I and/or Pvu I; in these cases we used buffer 3 of New England Biolabs and 0.1 mg/ml of BSA.

Digestion with Bae I was carried out with buffer 4 of New England Biolabs, to which 0.1 mg/ml of BSA and 20 μM end concentration of S-adenosylmethionine were added.

All the enzymes were inactivated after the reactions by incubation at 65° C. or 80° C., depending on the enzyme. In all cases the restriction cleavages were analyzed by agarose gel electrophoresis. We included control points without DNA but with the corresponding restriction buffers in all the amplification experiments.

In the experiments in which ligation proved necessary, the pEYFP-N1 plasmid was cleaved using BsmB I and EcoO109 I with buffer 3 of New England Biolabs during 8 hours, followed by purification using Qiagen columns and ligation in 1:4 proportion together with the hybridized oligonucleotides, in order to generate double strand DNA. After inactivating the ligase by heating to 65° C. during 20 min, the samples were used as amplification reaction templates with no further treatment, except were indicated otherwise.

Dephosphorylation was carried out with Antarctic phosphatase from New England Biolabs, adding its corresponding buffer and incubating at 37° C. for 15 min, followed by inactivation through incubation at 65° C. during 20 min. Following this treatment, the samples were precipitated with ethanol in the presence of 20 μg of glycogen as transporter, to eliminate the phosphatase buffer. After precipitation of the samples, the latter were resuspended in ligase buffer and used directly in the amplification experiments. Where indicated, the samples were treated with ATP and T4 polynucleotide kinase from New England Biolabs, in T4 ligase buffer at 37° C. during 30 min, and this enzyme was likewise inactivated through incubation at 65° C. during 20 min.

### Example 11

### Amplification Experiment

The amplification reactions were carried out in a volume of 25 μl, in A 1× buffer (50 mM Tris-HCl, pH 7.5, 10 mM MgCl₂, 5% glycerol, 1 mM DTT, 0.1 mg/ml BSA) supplemented with (NH₄)₂SO₄ to 20 mM and with an end concentration of 100 μM corresponding to each of the nucleotides: dCTP, dGTP, dTTP and dATP, [α-32P]dATP (1 μCi), 15 μg φ29 SSB (40 μM), 10 μg p6 (27 μM), 20 ng of DNA polymerase of bacteriophage φ29 (13 nM) and 20 ng of φ29 TP (26 nM), except where indicated otherwise, and the indicated amounts of vector or TP-DNA as template.

The reactions were started by adding the rest of the components to the template and TP (previously mixed), and were allowed to run for two hours at 22° C. The reactions were subsequently stopped by adding EDTA 10 mM and 0.1% SDS, as end concentrations. The samples were dried, resuspended in 50 μl of NaOH 0.5 M, and analyzed by 0.7% agarose gel electrophoresis in the presence of NaOH. We show the typical results of at least three independent experiments.

1. A genic construct adapted for the introduction of an exogenous nucleotide sequence, and which consists of at least a nucleotide sequence with the following three elements in the stated order:
a) at least a nucleotide sequence comprising a replication origin of the DNA of bacteriophage φ29, with a terminal extremity followed by:
b) at least one cleavage point of the nucleotide sequence,
c) at least a nucleotide sequence comprising a replication origin of the DNA of bacteriophage φ29 with an orientation opposite that of the nucleotide sequence in (a).

2. The genic construct according to the above claim, where the nucleotide sequences of the three elements are overlapped.

3.-5. (canceled)

6. The genic construct according to claim 1, where the genic construct is a circular vector.

7. (canceled)

8. The genic construct according to claim 1, where the replication origin of the DNA of bacteriophage φ29 corresponding to the nucleotide sequences of (a) and (b) are of the same extremity of the DNA of bacteriophage φ29.

9.-10. (canceled)

11. The genic construct according to claim 1, where the replication origin of the DNA of bacteriophage φ29 corresponding to the nucleotide sequence (a) and the replication origin of the DNA of bacteriophage φ29 corresponding to the nucleotide sequence (c) are of different extremities of the DNA of bacteriophage φ29.

12.-13. (canceled)

14. The genic construct according to claim 1, where the nucleotide sequence comprising the left replication origin of the DNA of bacteriophage φ29 contains between 65 and 72 nucleotides of the left extremity of the DNA of bacteriophage φ29.

15. (canceled)

16. The genic construct according to claim 1, where the nucleotide sequence comprising the right replication origin of the DNA of bacteriophage φ29 contains between 150 and 200 nucleotides of the right extremity of the DNA of bacteriophage φ29.

17. (canceled)

18. The genic construct according to claim 1, which moreover comprises at least one marker.

19.-21. (canceled)

22. The genic construct according to claim 1, comprising SEQ ID NO: 1, SEQ ID NO: 2, SEQ ID NO: 3 or a biologically equivalent variant.

23.-28. (canceled)

29. A DNA amplification method comprising at least the following steps:
a) obtainment of a linear DNA molecule comprising the DNA sequence destined for amplification and flanked on both extremities by:
i) the sequence of the left replication origin of the DNA of bacteriophage φ29, the terminal extremity of this origin being located in the linear DNA extremity; or by
ii) the sequence of the right replication origin of the DNA of bacteriophage φ29, the terminal extremity of this origin being located in the linear DNA extremity; or by
iii) the sequence of the right replication origin of the DNA of bacteriophage φ29 on one side and of the left replication origin on the other—the terminal extremity of these origins being located in the two extremities of the linear DNA;
b) amplification of the linear DNA sequence obtained in step (a).

30. The method according to claim 29, where use is made of a DNA polymerase, a terminal protein (TP), a single strand DNA binding protein (SSB) and a double strand DNA binding protein (DBP) in the amplification of step (b).

31. The method according to claim 30, where use is made of the proteins DNA polymerase, TP, p5 and p6 of bacteriophage φ29, or any bioequivalent variant of these proteins, in the amplification of step (b).

32. (canceled)

33. The method according to claim 29, where the linear DNA sequence obtained in step (a) presents phosphorylation of both 5′ extremities.

34. (canceled)

35. The method according to claim 29, where the sequence of the left replication origin of the DNA of bacteriophage φ29 contains between 67 and 72 nucleotides of the left extremity of the DNA of bacteriophage φ29.

36. (canceled)

37. The method according to claim 29, where the sequence of the right replication origin of the DNA of bacteriophage φ29 contains between 150 and 200 nucleotides of the right extremity of the DNA of bacteriophage φ29.

38. The method according to claim 29, where the sequence of the left replication origin of the DNA of bacteriophage φ29 contains between 65 and 200 nucleotides or between 67 and 72 nucleotides of the left extremity of the DNA of bacteriophage φ29, and the sequence of the right replication origin of the DNA of bacteriophage φ29 contains between 125 and 250 nucleotides or between 150 and 200 nucleotides of the right extremity of the DNA of bacteriophage φ29.

39. The method according to claim 29, where step (b) is carried out at a temperature of between 20 and 30° C.

40.-41. (canceled)

42. A kit for the amplification of an exogenous nucleotide sequence, consisting of at least a genic construct adapted for the introduction of an exogenous nucleotide sequence, and which consists of at least a nucleotide sequence with the following three elements in the stated order:
a) at least a nucleotide sequence comprising a replication origin of the DNA of bacteriophage φ29, with a terminal extremity followed by:
b) at least one cleavage point of the nucleotide sequence,
c) at least a nucleotide sequence comprising a replication origin of the DNA of bacteriophage φ29 with an orientation opposite that of the nucleotide sequence in (a); and a suitable buffer.

43.-47. (canceled)

48. The kit according to claim 42, where the nucleotide sequence comprising the left replication origin of the DNA of bacteriophage φ29 contains between 65 and 72 nucleotides of the left extremity of the DNA of bacteriophage φ29.

49. The kit according to claim 42, where the nucleotide sequence comprising the right replication origin of the DNA of bacteriophage φ29 contains between 150 and 200 nucleotides of the right extremity of the DNA of bacteriophage φ29.


SEQUENCE LISTING (15 sequences)

SEQ ID NO: 1 - DNA, 5833 bp, Artificial Sequence
  Vector pETORPHI
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgcatgcaggcctctgcagtcgacgggcccgggatccgatccaatcagtggctgtgcgacacagacgaagcgctaaaacgtgggattctgtgtcgttttatgttgttcattgacaaacctatctagtaggtctatagattgatatattaagtagtagtttccctccctgatcatggtatgccgaggatcgacggattatgtcgatattaggagaatggtatcatgtgagggtgggggcttactttaaagtagggtacagcgacaacatacaccatttccccattgaccgactatcttcgacaagaatctaacaactaaatcacgactatatacctatactatttattatcatcaatttgtcgaaaagggtagacaaactatcgtttaacatgttatactataatagaagtaaggtaataagacaaccaatcataggaggcacgagattgatctagatgcattcgcgaggtaccgagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 2 - DNA, 5666 bp, Artificial Sequence
  Vector pETORPHI68L
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgcatgcaggcctctgcagtcgacgggcccgggatccgatccaatcagtggctgtgcgacacagacgaagcgctaaaacgtgggattctgtgtcgttttatgttgttcattgacaaacctatctagtaggtctatagattgatatattaagtagtagtttccctccctgatcatggtatgccgaggatcgacggattatgtcgatattaggagaatggtatcatgtgagggtgggggcttactttaaagtaagcccccaccctcacatgataccattctcctaatatcgacataatccgtcgatcctcggcataattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - DNA, 5871 bp, Artificial Sequence
  Vector pETORPHIBae
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgcatgcaggcctctgcagtcgacgggcccgggatccgatccaatcagtggctgtgcgacacagacgaagcgctaaaacgtgggattctgtgtcgttttatgttgttcattgacaaacctatctagtaggtctatagattgatatattaagtagtagtttccctccctgatcatggtatgccgaggatcgacggattatgtcgatattaggagaatggtatcatgtgagggtgggggcttacttttctcctaattatttgactgtcgtatctgtcatcaaccaaaagtagggtacagcgacaacatacaccatttccccattgaccgactatcttcgacaagaatctaacaactaaatcacgactatatacctatactatttattatcatcaatttgtcgaaaagggtagacaaactatcgtttaacatgttatactataatagaagtaaggtaataagacaaccaatcataggaggcacgagattgatctagatgcattcgcgaggtaccgagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 4 - DNA, 3110 bp, Artificial Sequence
  Vector pUC57ORPHI
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatctcgtgcctcctatgattggttgtcttattaccttacttctattatagtataacatgttaaacgatagtttgtctacccttttcgacaaattgatgataataaatagtataggtatatagtcgtgatttagttgttagattcttgtcgaagatagtcggtcaatggggaaatggtgtatgttgtcgctgtaccctactttaaagtaagcccccaccctcacatgataccattctcctaatatcgacataatccgtcgatcctcggcataccatgatcagggagggaaactactacttaatatatcaatctatagacctactagataggtttgtcaatgaacaacataaaacgacacagaatcccacgttttagcgcttcgtctgtgtcgcacagccactgatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 68 bp, Bacteriophage phi 29
aaagtaagcccccaccctcacatgataccattctcctaatatcgacataatccgtcgatcctcggcat

SEQ ID NO: 6 - DNA, 72 bp, Artificial Sequence
  Oligonucleotide with 68 nucleotides from the left end of bacteriophase Phi-29 DNA at the 3′ end and the cohesive end of EcoRI restriction site at the 5′ end
aattatgccgaggatcgacggattatgtcgatattaggagaatggtatcatgtgagggtgggggcttacttt

SEQ ID NO: 7 - DNA, 72 bp, Artificial Sequence
  Oligonucleotide with 68 nucleotides from the left end of bacteriophage Phi-29 DNA at the 3′ end and the cohesive end of BsmBI restriction site at the 5′ end
atccatgccgaggatcgacggattatgtcgatattaggagaatggtatcatgtgagggtgggggcttacttt

SEQ ID NO: 8 - DNA, 71 bp, Artificial Sequence
  Oligonucleotide with 68 nucleotides from the left end of bacteriophage Phi-29 DNA at the 3′ end and the cohesive end of Eco109I restriction site at the 5′ end
gccatgccgaggatcgacggattatgtcgatattaggagaatggtatcatgtgagggtgggggcttacttt

SEQ ID NO: 9 - DNA, 38 bp, Artificial Sequence
  Forward oligonucleotide used to generate BaeI restriction site sequence inserted in the DraI restriction site of pETORPHI
tctcctaattatttgactgtcgtatctgtcatcaacca

SEQ ID NO: 10 - DNA, 38 bp, Artificial Sequence
  Reverse oligonucleotide used to generate BaeI restriction site sequence inserted in the DraI restriction site of pETORPHI
tggttgatgacagatacgacagtcaaataattaggaga

SEQ ID NO: 11 - DNA, 5368 bp, Artificial Sequence
  Vector pET28b
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 12 - DNA, 191 bp, Bacteriophage Phi29
aaagtaagcccccaccctcacatgataccattctcctaatatcgacataatccgtcgatcctcggcataccatgatcagggagggaaactactacttaatatatcaatctatagacctactagataggtttgtcaatgaacaacataaaacgacacagaatcccacgttttagcgcttcgtctgtgtcgca

SEQ ID NO: 13 - DNA, 194 bp, Artificial Sequence
  Bacteriophage Phi-29
aaagtagggtacagcgacaacatacaccatttccccattgaccgactatcttcgacaagaatctaacaactaaatcacgactatatacctatactatttattatcatcaatttgtcgaaaagggtagacaaactatcgtttaacatgttatactataatagaagtaaggtaataagacaaccaatcataggagg

SEQ ID NO: 14 - DNA, 4733 bp, Artificial Sequence
  pEYFP-N1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 15 - DNA, 4733 bp, Artificial Sequence
  pEYFP-N1Bsm
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgcgtctctggataaccgtattaccgccatgcat